(19) United States
(12) Patent Application Publication
(10) Pub. No.: US 2026/0151223 A1
(43) Pub. Date: Jun. 4, 2026

(54) IMPROVED STEM OF THE PROTHESIS FOR THE RECONSTRUCTION OF THE OSSICULAR CHAIN

(71) Applicant: Giuseppe MALAFRONTE, Napoli (IT)

(72) Inventor: Giuseppe MALAFRONTE, Napoli (IT)

(21) Appl. No.: 19/132,487

(22) PCT Filed: Jan. 9, 2024
(86) PCT No.: PCT/IT2024/050002

§ 371 Date: May 23, 2025

(30) Foreign Application Priority Data
Jan. 17, 2023 (IT) 102023000000570

(51) Int. Cl. A61B 17/17; A61B 17/84; A61B 17/88; A61F 2/06; A61F 2/08; A61F 2/18; A61F 2/20; A61F 2/24; A61F 2/30; A61F 2/46; A61F 2/966

(52) U.S. Cl. CPC A61F 2/18; A61L 27/047; A61L 27/06; A61L 27/14; A61L 27/3612; A61F 2002/183; A61F 2220/0033; A61F 2230/0069

(57) ABSTRACT

An improved TORP for the reconstruction of the ossicular chain provides, in combination: a head (C) which is formed by a single cartilaginous block:—a synthetic stem (A) of cylindrical shape which is provided, on both sides, with a respective blunt tip (A1, A2); a cartilaginous base (B) having the same thickness as the head (C) but with a smaller surface.

Said improved TORP is configured in such a way as to be able to position, respectively, said head (c) under the tympanic membrane (D) and said base (B) above the plate (E) of the stapes (s); thus obtaining that during the healing process, the perichondrium of the head (c) is welded to the tympanic membrane (D) and the perichondrium of the base (B) is welded to the mucosa of the plate (E).

The present invention concerns the surgical sector, with particular reference to ear pathologies.

The tympanic cavity is an air cavity that communicates with the nasal cavities thanks to the Eustachian tube. The tympanic cavity contains three ossicles: hammer, incus and stapes, which make up the so-called “ossicular chain”. The aim of the ossicular chain is to transport sound from the tympanic membrane to the cochlea. In the latter, the mechanical energy of sound is transformed into electrical energy, that is, into electrical pulses which, through the auditory nerve, are sent to the brain. A damage to the ossicular chain causes a reduction in hearing ability, called transmission hearing loss.

The operation to reconstruct the ossicular chain is called “ossiculoplasty”. Based on the broken ossicles we have two types of ossiculoplasty:
partial ossiculoplasty, when only one ossicle is damaged, i.e. the incus;
total ossiculoplasty, when two small ossicles are damaged, namely the incus and the superstructure of the stapes.

The prosthesis used in partial ossiculoplasty is called PORP (Partial Ossicular Replacement Prosthesis). The PORP is placed between the tympanic membrane and the stapes capital and replaces the damaged incus. The prosthesis used in total ossiculoplasty is called TORP (Total Ossicular Replacement Prosthesis). The TORP is placed between the tympanic membrane and the plate of the stapes and replaces the damaged incus and the superstructure of the stapes.

It is known from U.S. Pat. No. 4,641,651, a TORP comprising a head which is formed from a cartilaginous block and a synthetic stem of cylindrical shape which is provided with a tip at one end.

A better understanding of the invention will be provided by the following detailed description and with reference to the attached figures which illustrate some already known technical solutions, as well as a preferred embodiment of the invention, illustrated and described here by way of example and not by way of limitation.

In the drawings:

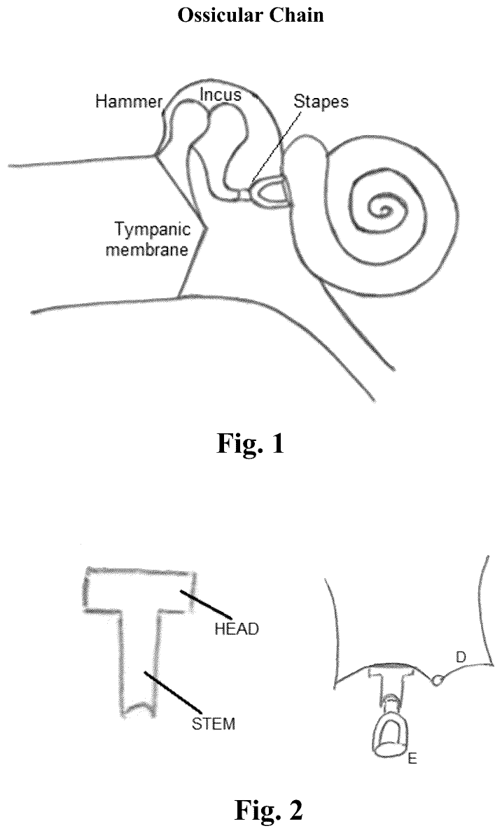

FIG. 1 schematically shows the ossicular chain of a human ear;

FIG. 2 shows respectively: on the left the shape of a synthetic PORP of a known type, and on the right its positioning between the tympanum and the stapes;

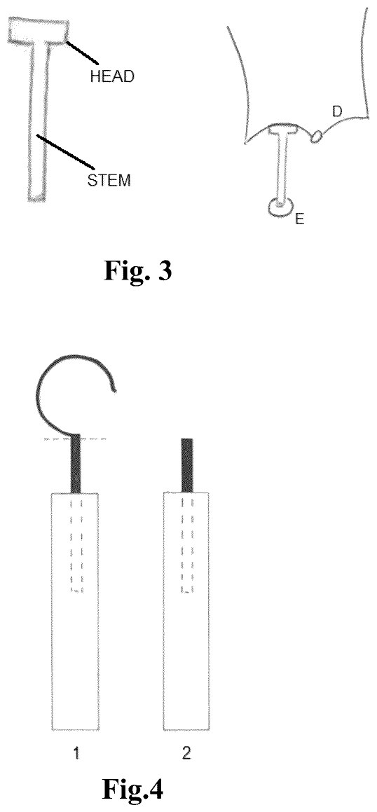

FIG. 3 shows respectively on the left the shape of a synthetic TORP of a known type and on the right its positioning between the tympanum and the plate of the stapes;

FIG. 4 shows a prosthesis for stapes surgery (1) and the same prosthesis modified (2) to create a semi-synthetic static TORP of a known type;

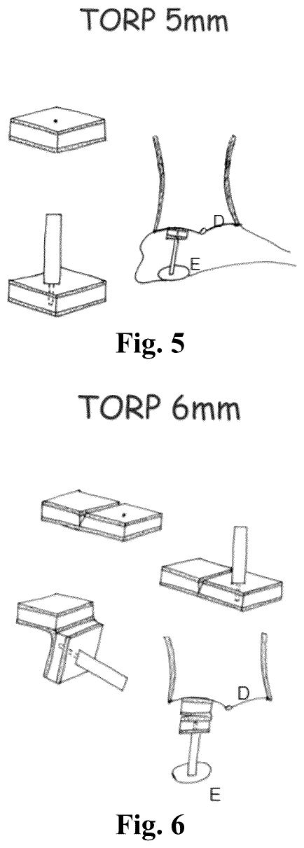

FIG. 5 illustrates how a 5 mm long semi-synthetic TORP of known type is made and its positioning for total ossiculoplasty;

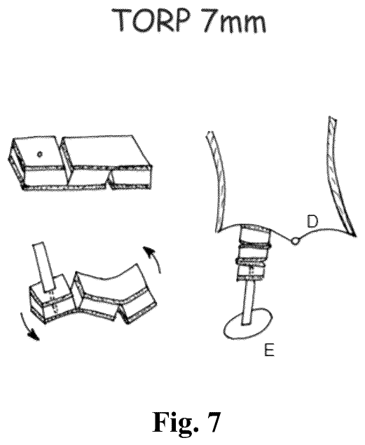

FIGS. 6 and 7, similar to FIG. 5, are related to a semi-synthetic TORP of 6 mm and 7 mm length, respectively;

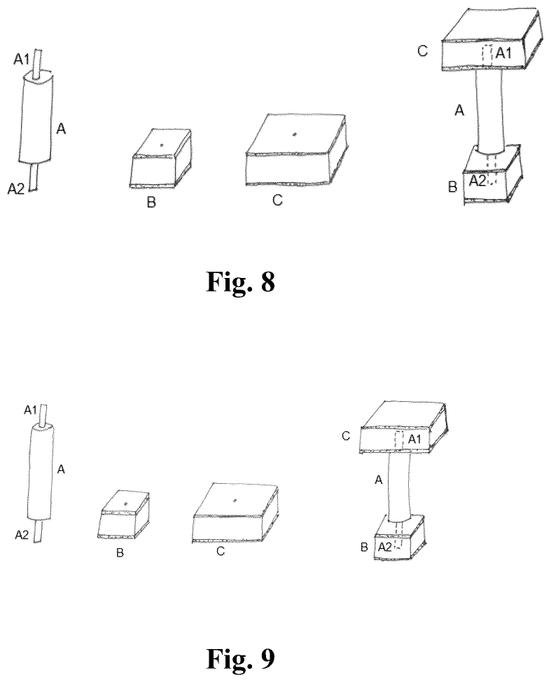

FIG. 8 illustrates how an improved TORP according to the present invention is constructed having an overall length of 5 mm;

FIG. 9 illustrates how to construct an improved TORP according to the present invention having an overall length of 6 mm;

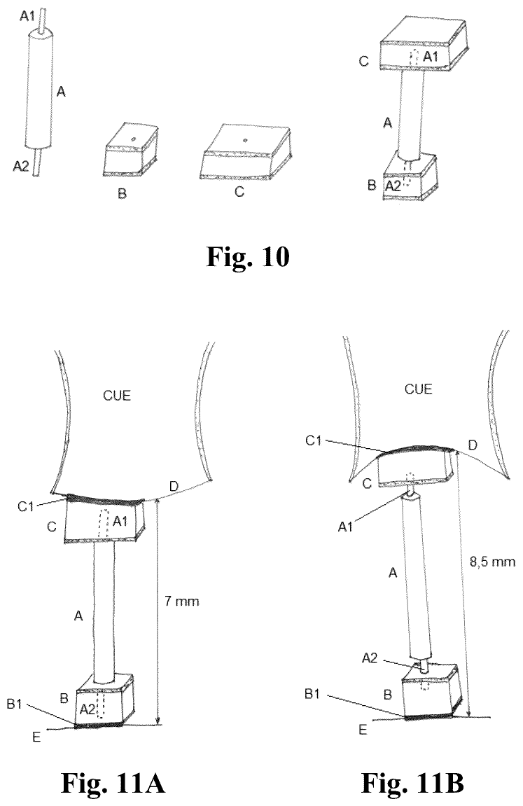

FIG. 10 illustrates how to construct an improved TORP according to the present invention having an overall length of 7 mm;

FIGS. 11A and 11B illustrate the invention placed between the tympanum and the plate for the total reconstruction of the ossicular chain (incus and superstructure of the damaged staples), as well as its behavior in the event of variations in atmospheric pressure;

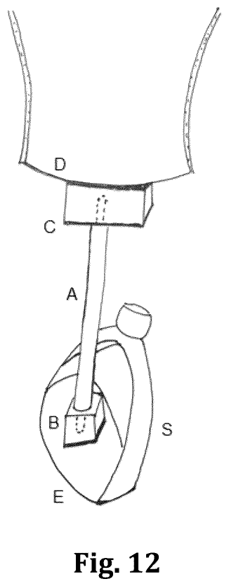

FIG. 12 illustrates the invention placed between the tympanum and the plate of an intact stapes for the reconstruction of the ossicular chain, when only one ossicle (incus) is damaged.

TORPs currently in use are synthetic in nature. They can be made of gold, platinum, hydroxyapatite or titanium. Each of these TORPs consists of a flat head with a diameter of approximately 2.5 mm placed at the end of a cylindrical stem (FIG. 3). Usually the distance between the tympanic membrane and the plate varies from 5 to 7 mm, in rare cases it is 8 mm. TORPs most used in the reconstruction of the ossicular chain have a total length of 5 or 6 or 7 mm.

The drawbacks of the current TORPs are essentially four:
1—They do not have a good anchoring to the tympanic membrane;
2—They do not have a stable anchoring to the plate of the stapes;
3—They are unable to change their length based on the movements of the tympanic membrane;
4—They do not have a high biocompatibility in hostile environments.

The reduction in atmospheric pressure (air travels, mountain excursions) determines an increase in the pressure present in the tympanic cavity, with a displacement of the tympanic membrane of 1 or 1.5 millimeters towards the outside. The prostheses on the market are static prostheses, as they cannot change their length, they easily lose contact with the tympanic membrane and the plate and become dislocated. Similarly, an increase in atmospheric pressure (underwater activity) or tubal dysfunctions determine a reduction in the pressure present in the tympanic cavity, with an inward movement of the tympanic membrane. The increase in tension between the tympanum and the synthetic prosthesis causes the extrusion of the prosthesis itself. The extrusion, or dislocation, of this prosthesis determines the reappearance of the transmission hear loss.

These are the main reasons why, in the long term, the extrusion or dislocation rate of synthetic TORP is high (35-55%). To solve or at least reduce these reappearance rates of transmission hear loss, in 2007 the same Applicant proposed and published a new type of TORP called “semi-synthetic TORP” (hereinafter also called TORPss).

This TORPss is made by assembling a cylindrical titanium and polytetrafluoroethylene stem with an autologous cartilaginous head. To create the stem of this TORPss, the base of the hook of a prosthesis is cut—in fact of a known type (made of titanium and polytetrafluoroethylene)—for stapes surgery (FIG. 4A) with a total length of 7 mm.

This results in a cylindrical stem made of titanium and polytetrafluoroethylene with a diameter of 0.4 mm and a length of 4 mm. This stem is provided with a blunt titanium tip with a diameter of 0.2 mm and 1 mm in length (FIG. 4B). The blunt tip is completely inserted into the cartilaginous head (FIG. 5). In particular, the head of the TORPss can be made of one, two or three blocks of tragus cartilage held together by a perichondrium hinge (FIGS. 5, 6, 7). The tragus cartilage is ideal for building blocks as it is flat and has a thickness of 1 mm. The 5 mm long TORPss has a head made of a single 2.5×2.5 mm square block of cartilage covered on both sides by perichondrium. In the center of the block, only in the perichondrium, a hole is made with an insulin needle. The blunt tip of the stem is then inserted into the hole in the perichondrium: by exerting a small pressure the tip penetrates completely into the cartilage. This results in a 5 mm long TORPss (FIG. 5). The assembly of the head cartilaginous block with the stem appears to be very stable. The total reconstruction of the ossicular chain is obtained by placing the cartilaginous head under the tympanic membrane and the base of the synthetic stem above the plate (FIG. 5).

The 6 mm long TORPss is obtained by assembling the stem with two cartilaginous blocks, held together by a perichondrium hinge (FIG. 6). Similarly, the 7 mm long TORPss is obtained by assembling the stem with three cartilaginous blocks, held together by a perichondrium (FIG. 7).

However, it is necessary to note that the TORPss has only solved two of the four drawbacks of the synthetic TORP, as in fact:
1—The head of the TORPss has a good anchoring to the tympanum as the perichondrium of the cartilaginous block in contact with the tympanic membrane, during the healing process, is welded to the tympanic membrane and does not detach from the tympanum even in the presence of pathologies;
2—The base of the TORPss does not have a good anchoring to the plate of the stapes;
3—The TORPss is a static prosthesis, not being capable to modify its length based on the movements of the tympanic membrane;
4—The TORPss has a high biocompatibility in a hostile environment as the cartilaginous head avoids contact of the synthetic stem with the tympanic membrane and prevents its extrusion in the presence of pathologies.

The percentage of extrusion of the TORPss and its dislocation from the tympanic membrane is very low, however the problem of the dislocation of the base of the stem from the plate of the stapes remains.

In essence, drawbacks of the TORPss are:
1—the construction of the cartilaginous head of the TORPss, which is simple in the 5 mm long TORPss as it is formed by a single cartilaginous block, is more difficult in the 6 or 7 mm long TORPss as it is formed by two or three cartilaginous blocks held together by a cartilaginous hinge: its construction requires a lot of experience from the operator.
2—it is a static prosthesis, not being capable to increase its length when the distance between the membrane and the plate increases during variations in atmospheric pressure.
3—the lack of anchoring of the base of the synthetic stem to the plate of the stapes, and the inability to increase its length have the disadvantage that the stem, not anchored, can easily dislocate from the plate—especially during variations in atmospheric pressure—causing the reappearance of a transmission hearing loss.

Main purpose of the present invention is to overcome these drawbacks, providing an innovative improved TORP compared to the traditional TORPss described above.

The improved TORP according to the invention is characterized in that it provides, in combination:
a head which is formed by a cartilaginous block covered by perichondrium (1 mm thick and a surface area of 2.5×2.5 mm) the construction of which is very simple (FIGS. 8, 9, 10);
a new synthetic stem with a cylindrical shape which is provided, on both sides, with a respective blunt titanium tip 1 mm long and 0.2 mm in diameter (FIGS. 8, 9, 10);
a base which is formed by a cartilaginous block covered by a perichondrium (1 mm thick and by a surface area of 0.8×0.8 mm (FIGS. 8, 9, 10);

in which the different length of the semi-synthetic prosthesis is obtained by providing cylindrical synthetic stems of different lengths.

According to the present invention, the tip (A1) of one end of the stem (A) is entirely inserted into the head (C) of the prosthesis which is formed by a cartilaginous block covered by a perichondrium, while the tip (A2) of the opposite end of the stem (A) is entirely inserted into the base (B) of the prosthesis, which is formed by a cartilaginous block smaller than that of the head (FIGS. 8, 9, 10).

Therefore, with reference to FIGS. 8, 9 and 10, the new improved TORP consists of:
a cartilaginous head 1 mm thick and with a surface area of 2.5×2.5 mm;
a synthetic cylindrical stem (in gold, platinum, titanium and polytetrafluoroethylene), with a diameter of 0.4 mm and having on each of the two ends a blunt tip 1 mm long and with a diameter of 0.2 mm, said stem having a length equal to:
   o 3 mm for the 5 mm long TORP (FIG. 8);
   o 4 mm for the 6 mm long TORP (FIG. 9);
   r 5 mm for the 7 mm long TORP (FIG. 10);
a cartilaginous base 1 mm thick and with a surface area of 0.8×0.8 mm.

In the reconstruction of the ossicular chain, the head of the new prosthesis according to the present invention is placed under the tympanic membrane and its base above the plate of the stapes (FIG. 11A).

Advantageously, during the healing process, the perichondrium of the head is welded to the tympanic membrane and the perichondrium of the base is welded to the mucosa of the plate. For this reason, the improved TORP that is described is very stable as it has two solid anchors: one with the tympanum and the other with the plate. On the contrary, the traditional TORPss is less stable as it has only one anchor: the one with the tympanum.

Furthermore, the TORP according to the invention is simpler to construct than the traditional TORPss, and allows for a more stable reconstruction of the ossicular chain, even in the event of variations in atmospheric pressure which cause an increase in the distance between the tympanic membrane and the plate. In these events, in fact, the blunt tips at the two ends still remain partially inserted in the respective holes of the head and the base, without causing dislocations (FIGS. 11A and 11B).

Finally, FIG. 11a shows the semi-synthetic dynamic prosthesis which is the object of the present invention, placed between the tympanic membrane (D) and the plate (E).

FIG. 11b shows the behavior of the aforementioned prosthesis in the event of a displacement of the tympanic membrane (D) in the external auditory canal (CUE) with an increase in the distance between the tympanic membrane (D) and the plate (E), with a partial protrusion of the tip (A1) of the stem (A) of the prosthesis head (C) and a partial protrusion of the tip (A2) of the stem (A) from the base of the prosthesis (B).

FIG. 12 shows the interposition of the semi-synthetic dynamic prosthesis between the tympanic membrane (D) and the plate (E) of the intact stapes (s).

### Legend

A: synthetic stem 3 or 4 or 5 millimeters long
A1: tip of the stem (1 mm long) to be inserted into the head of the prosthesis
B: base of the prosthesis formed by a cartilaginous block with perichondrium (1 mm thick; 0.8×0.8 mm in surface)
C: head of the prosthesis formed by a cartilaginous block with perichondrium (1 mm thick; 2.5×2.5 mm in surface)
A: synthetic stem of the prosthesis
A1: tip of the stem completely inserted into the head
A2: tip of the stem completely inserted into the base
B: base of the prosthesis made of cartilage covered by perichondrium
B1: perichondrium of the base welded to the plate E
C: head of the prosthesis made of cartilage covered by perichondrium
C1: perichondrium of the head welded to the tympanic membrane D
D: tympanic membrane
E: plate
CUE: external auditory canal

1-5. (canceled)

6. An improved Total Ossicular Replacement Prosthesis (TORP) for reconstruction of an ossicular chain, said improved TORP comprising:
a head formed by a single cartilaginous block and is provided with a first hole;
a base formed by another cartilaginous block, provided with a second hole, comprising a same thickness as the head but with a smaller surface;
a synthetic stem of cylindrical shape which comprising two sides and two blunt tips, wherein each blunt tip is provided on one side of said two sides, such that a first blunt tip is on a first side of the synthetic stem and a second blunt tip is on a second side of the synthetic stem that is opposite to said first side, wherein said two blunt tips are inserted, respectively, said first hole of the head and said second hole of the base;
wherein said improved TORP is configured in such a way as to be able to position, respectively, said head under a tympanic membrane and said base above a plate of a stapes;
such that, during a healing process, a perichondrium of the head is welded to the tympanic membrane and a perichondrium of the base is welded to a mucosa of the plate.

7. The improved TORP according to claim 6, wherein the first blunt tip of said first side of the synthetic stem is configured to be completely inserted into said head which is formed by said single cartilaginous block covered by said perichondrium, and wherein the second blunt tip of the second side of the synthetic stem is configured to be completely inserted into said base which is formed by said another cartilaginous block smaller than that of the head.

8. The improved TORP according to claim 6, wherein the improved TORP is 5 mm long or 6 mm long or 7 mm long and wherein
said head comprises a thickness of 1 mm and a surface area of 2.5×2.5 mm;
said synthetic stem comprises
gold, or platinum, or titanium, or titanium and polytetrafluoroethylene, with a diameter of 0.4 mm, and
wherein each blunt tip of said first blunt tip and said second blunt tip comprise
a length of 1 mm and a diameter of 0.2 mm, and
a length comprising
3 mm for the 5 mm long improved TORP,
4 mm for the 6 mm long improved TORP,
5 mm for the 7 mm long improved TORP;
said base comprises a thickness of 1 mm and a surface area of 0.8×0.8 mm.

9. The improved TORP according to claim 6, wherein in an event of variations in atmospheric pressure, said two blunt tips at the two sides remain in any case at least partially inserted in, respectively, said first hole of the head and said second hole of the base, without causing dislocations.

10. The improved TORP according to claim 6, wherein the synthetic stem further comprises a length greater than a height of the stapes, configured to be used to reconstruct the ossicular chain both only when an incus is injured, and when the incus and a superstructure of the stapes are injured.
